(19) United States
(12) Patent Application Publication
(10) Pub. No.: US 2022/0000803 A1
(43) Pub. Date: Jan. 6, 2022

(54) COMPOSITIONS CONTAINING FORMALDEHYDE AND ORGANIC ACID FOR PREVENTION OF AFRICAN SWINE FEVER

(71) Applicant: KEMIN INDUSTRIES, INC., Des Moines, IA (US)

(72) Inventors: Andrew Yersin, Urbandale, IA (US); Hai Meng Tan, Bukit Timah (SG); Alfred Eng Kheng Chua, Tanamera (SG)

(21) Appl. No.: 16/920,512

(22) Filed: Jul. 3, 2020

(51) Int. Cl. A61K 31/115; A61P 31/20; A61K 31/191; A61K 31/19; A61K 9/00; A23K 20/105; A23K 20/121; A23K 50/30

(52) U.S. Cl. CPC A61K 31/115; A61P 31/20; A61K 31/191; A61K 31/19; A23K 50/30; A61K 9/0053; A23K 20/105; A23K 20/121; A61K 9/0095

(57) ABSTRACT

The present invention relates to compositions and methods to prevent the incidence of African Swine Fever (ASF) in swine. Another aspect of the present invention relates to the administration of formaldehyde and at least one organic acid, preferably in the animal's feed or drinking water. The invention is unexpectedly effective in controlling the spread of the ASP virus but also keeping the animal feed virus-free for up to 7 days.

### CROSS-REFERENCE TO RELATED APPLICATIONS

This application claims the benefit of priority to U.S. Provisional Patent Application No. 62/870,560, filed Jul. 3, 2019, entitled “COMPOSITIONS CONTAINING FORMALDEHYDE AND ORGANIC ACID FOR PREVENTION OF AFRICAN SWINE FEVER,” the disclosure of which is hereby incorporated by reference in its entirety.

### BACKGROUND OF THE INVENTION

African swine fever (ASF) is a highly contagious hemorrhagic viral disease of domestic and wild pigs, which is responsible for serious economic and production losses. ASF resembles classical swine fever (CSF) (hog cholera) so closely that laboratory tests are required to differentiate them. ASF is caused by a unique virus of the Asfarviridae family, and which only infects domestic and wild pigs and a variety of soft-bodied ticks of the genus Ornithodoros. The ticks transmit it through all stages of their life cycle and perpetuate the disease. ASF is currently present in wild and/or domestic pigs in the regions of China, Asia, Europe, and Africa.

Pigs and their close relatives, boars and hogs, are the only natural host of the ASF virus, meaning that the virus does not harm humans or other animals. Contamination generally occurs via direct contact with tissue and bodily fluids from infected or carrier pigs. It also spreads through transport and consumption of contaminated food products, and some cases have originated from failure to comply with biosecurity standards by feeding waste food to domestic pigs.

ASF can affect pigs of any age. Clinical signs and mortality rates can vary according to the virulence of the virus and the type/species of pig. Acute forms of ASF are characterized by high fever, depression, anorexia and loss of appetite, hemorrhages in the skin (redness of skin on ears, abdomen and legs), abortion in pregnant sows, cyanosis, vomiting, diarrhea and death within 6-13 days. The mortality rates of pigs acutely infected with ASF may be as high as 100%. Subacute and chronic forms of ASF produce less intense clinical signs that may be expressed for longer periods of time, but with mortality rates still ranging from 30-70%.

ASF has not yet reached or been reported in pigs in the United States. As there is currently no vaccine or treatment for the prevention and control of ASF, the prevention of ASF in countries free of the disease depends on implementation of appropriate import policies and biosecurity measures, ensuring that neither infected live pigs nor pork products are introduced into areas free of ASF. This includes ensuring proper disposal of waste food from aircraft, ships or vehicles coming from affected countries and policing illegal imports of live pigs and pork products from affected countries. However, during outbreaks of ASF in affected countries, the control of ASF can be very difficult.

There is therefore a need in the industry for an effective means of identifying, preventing, and treating ASF.

### SUMMARY OF THE INVENTION

The present invention relates to the use of formaldehyde in combination with an organic acid that can strongly inhibit viricidal activity of African swine fever (ASF) when applied to animal feed or in drinking water. The inventors have determined hypothesized that the unique combination of formaldehyde in conjunction with an organic acid disrupts the viral envelope of the ASF virus (ASFV), resulting in successful killing of the virus while also keeping the feed virus-free for at least seven days, and thus potentially reducing infection in the animal via feed and water as vectors.

### BRIEF DESCRIPTION OF THE FIGURES

FIG. 1 is a graph illustrating OIE Realtime PCR method confirming positive HADs as set forth in Example 1.

FIG. 2 is a sequencing analysis demonstrating that Nam Dinh and Ha Nam belong to genotype II as set forth in Example 1.

FIG. 3 is a graph illustrating Realtime PCR based on OIE protocol as set forth in Example 1.

FIG. 4 is a graph illustrating HAD50 data as set forth in Example 1.

FIG. 5A-5F are graphs illustrating the mean of Cq value following doses of ASFV exposure at 30 min (5A), 1 h (5B), 2 h (5C), 3 h (5D), 12 h (5E), and 24 h (5F).

### DETAILED DESCRIPTION OF THE PREFERRED EMBODIMENT

The invention relates to the use of formaldehyde in combination with an organic acid for treatment of animal feed to lower the incidence and/or prevent African swine fever (ASF) via feed and water as vectors. When used on animal feed and or drinking water, the unique combination of formaldehyde and organic acid results in both viral hemolysis as well as a decrease of ASF viral load. When only a combination of organic acids is used, only a decrease of ASF viral load is observed.

The present invention may be used to reduce any type or form of African swine fever (ASF) disease in swine caused by the African swine fever virus (ASFV), a large double stranded DNA virus in the Asfarviridae family, via feed and water as vectors

According to at least one embodiment of the invention, pigs or other swine are provided through their food or water source a composition containing one or more forms of formaldehyde and organic acid. As used herein, the term “formaldehyde” is intended to include any form of formaldehyde including, but not limited to, the hydrate methanediol that is formed when formaldehyde is dissolved in water, as well as derivatives thereof, such as the linear polymer paraformaldehyde and the cyclic trimer metaformaldehyde (1,3,5-trioxane), as well as urea formaldehyde

The formaldehyde is combined with at least one organic acid which acts as an antimicrobial and enables the reduction of virus by the formaldehyde and other ingredients. Any organic acid will work for this purpose so long as it is safe for ingestion and compatible with the formaldehyde and other ingredients used in the formulation. Suitable organic acids for this purpose include small to medium chain organic acids that include fatty, volatile fatty, lipophilic, weak, or carboxylic acids, as well as derivatives thereof. These include, but are not limited to, acetic acid, propionic acid, butyric acid, citric acid, malic acid, tartaric acid, oxalic acid, lactic acid, formic acid, and benzoic acid. According to at least one embodiment of the present invention, propionic acid and or formic are preferred organic acids.

In addition to formaldehyde and at least one organic acid, one or more emulsifiers/surfactants may also be applied to the feed. Suitable emulsifiers for this purpose include, but are not limited to, soya lecithin, glycerin monostearate, potassium stearate, calcium stearoyl lactylate (CSL), DATEM, glyceryl monostearate, mono propylene glycol, SPAN 80, sodium stearoyl lactylate (SSL), Tween, sodium stearate, glycerol triacetate, sugar esters, non-dairy creamer, calcium stearate, polyglycerol polyricinoleate (PGPR), lecithin, mono and diglycerides, monoglyceride derivatives, polyglycerol esters (PGE), propylene glycol esters (PGMS), sucrose esters, and sorbitan esters and polysorbates. Preferred emulsifiers for this purpose include Tween 80 and propylene glycol. If included, the emulsifier(s) should be added to the feed in an amount of from about 0.001-10.0% by weight.

The feed compositions of the invention should preferably include at least about 40 kgs of formaldehyde/metric ton of feed, at least about 10 kgs of organic acid/metric ton of feed and, if included, at least about 6 kgs of emulsifier/metric ton of feed.

In one embodiment of the invention, the composition includes the commercial formulation Sal CURB® RM E Liquid which contains about 33% formaldehyde and 10% propionic acid in a solution of coloring agents, with a pH of 4.6-5.6, and having a typical application of 2.0-2.5 kg/T of feed. In another embodiment, the composition includes the commercial formulation Sal CURB® F2 Dry, which contains about 4% formaldehyde, about 53% formic, 12% propionic acid, 4% Tween 80, 3% propylene glycol, and pinene alpha (0.35%) and thymol (0.15%) as flavors. In another embodiment, the composition includes the commercial formulation Sal CURB® K2 Liquid, which contains about 55% formic, 15% anhydrous ammonia, 10% propionic acid, 10 lactic acid, 1% Tween 80, and 1% BHA.

The compositions of the invention may optionally include other ingredients, such as antioxidants, so long as the other ingredients are compatible with the formaldehyde and organic acid. Antioxidants are well known in the art and include, but are not limited to, vitamin A, vitamin C, vitamin E, beta-carotene, lycopene, lutein, selenium, manganese, zeaxanthin, flavonoids, flavones, catechins, polyphenols, and phytoestrogens. In one embodiment, the composition includes butylated hydroxyanisole to prevent oil oxidation.

The compositions of the invention can further include other ingredients or compounds that may be beneficial for pigs including, but not limited to, carbohydrate, protein, fat and oil, vitamins, minerals, probiotics, medicines, flavors, colors, etc. The compositions may also be combined with a pharmaceutically acceptable carrier that may include one or more carriers or excipients, such as fillers, diluents, binders, lubricants, and disintegrants. Such ingredients and their relative amounts to be included are well known to persons skilled in the art.

While the compositions of the invention are described in particular for administration in the animal's feed, the compositions may likewise be administered in the animal's water source.

According to at least one embodiment, the ingredients of the formulation may be combined by simply mixing at room temperature (25-30° C.) with or without agitation. The ingredients of the invention can either be mixed sequentially or can be added all at once to achieve the unique composition of the invention. In preferred embodiments the ingredients are mixed with agitation to improve miscibility. The composition can in turn be simply combined with the animal feed prior to administration to the animals. Animal feed formulations may be provided to the animals through any convention means well known to persons skilled in the art, include but not limited to top-dress, mixed in by hand, pelleted, mixed with crumbles, etc.

Once combined with the animal feed, the composition of the invention is preferably provided to the animals ad libitum, and preferably for a time period of 7 days or more and optimally through the various feeding periods (nursery, grower, finisher, gestation, lactation, farrowing) of the swine.

The following examples are offered to illustrate but not limit the invention. Thus, it is presented with the understanding that various formulation modifications as well as method of delivery modifications may be made and still are within the spirit of the invention.

### EXAMPLE 1

### Viricidal Activity of Sal Curb® Products

I. Information of ASF Strain Used in this Study
ASFVs isolated from died pigs in Ha Nam and Nam Dinh provinces in 2019

1.1. HAD test: Positive

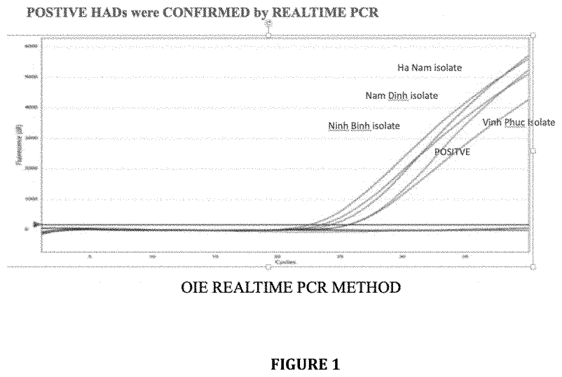

1.2. Real-time PCR: Positive (FIG. 1)

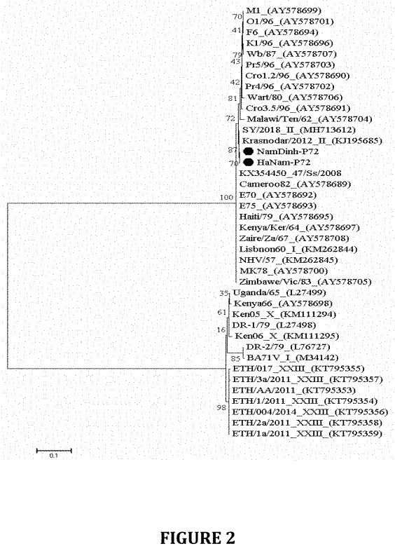

1.3. Sequencing analysis (Based on genotyping primer p72 U/D—OIE protocol): Positive. Nam Dinh and Ha Nam belong to genotype II (FIG. 2)

1.4. HAD50:

TABLE 1

| | HAD50/ml | | |
|---|---|---|---|
| ASF Isolates | First | Second | Third |
| Ha Nam | 10^{5.9} | 10⁶ | 10⁶ |
| Nam Dinh | 10^{5.7} | 10^{5.9} | 10^{5.7} |

### Conclusion:

Ha Nam isolate was selected for Examination of Kemin (Sal Curb) Products inhibiting viricidal activity. This isolate is genotype II.

### II. Experiment Design

Feed model: complete swine feed, negative for ASFV.
Treatment of the feed with Kemin products before contamination with ASFV
To ensure a homogeneous spread of the product on the feed, 200 g of complete feed will be treated with Sal CURB RM E Liquid, Sal CURB K2 Liquid and Sal CURB F2 Dry (3.0 kg/T).
Number of samples: 3 sampling time points*3 samples/time point*5 treatments
(a) Sampling timepoint 1: 1 day post contamination (DPC)
(b) Sampling timepoint 2: 3 DPC
(c)Sampling timepoint 3: 7 DPC (end of experiment)
Negative control (No ASFV added)
Positive control (complete feed containing 1×10⁵ (HAD50) of ASFV)
Treatment 1: positive control treated with Sal CURB RM E Liquid
Treatment 2: positive control treated with Sal CURB F2 Dry
Treatment 3: positive control treated with Sal CURB K2 liquid

### Virus/Feed Contamination Methods:

Use 5 g of feed in 50 mL mini bioreactor tubes (Corning Inc., Corning, N.Y.); with vented caps to allow exchange of temperature and humidity between the interior and exterior of the tube
Spike the feed with 100 μL of MEM (minimum essential media, Gibco, ThermoFisher Scientific, Waltham, Mass., US) containing 1×10⁵ (HAD50) of ASFV.
Negative control: 5 g of feed with 100 μl sterile MEM medium/sterile PBS
Vortex the samples for 10 seconds following the addition of the virus inoculum
Incubation of the samples at 20° C. and ˜60% RH (as per lab conditions) for 7days

### Processing of Samples:

At the different sampling time points (i.e. DPC), process the samples for virus isolation.
Resuspend each sample in 15 ml sterile PBS
Vortex for 10 seconds and centrifuge (only to remove feed particles)
Collect supernatant and centrifuge for 10 000 g for 10 minutes.
Use supernatant for virus isolation according to SOP and for virus titration.

### References:

Dee et al. (2018), Survival of viral pathogens in animal feed ingredients under transboundary shipping models. PLoS ONE 13(3): e0194509.

### III. Methods

1. Virus isolation and HAD test: OIE protocol
2. Conventional PCR and real-time PCR: OIE protocol

### IV. Results

### 4.1 Conventional PCR Using PPA1 and PPA2

### Note:

No 1. positive control treated with Sal CURB RM E Liquid

No 2. positive control treated with Sal CURB F2 Dry

No3. positive control treated with Sal CURB K2 liquid

No 4. Negative control (No ASFV added, only feed)

No 5. Positive control: complete feed containing 1×10⁵ (HAD50) of ASFV

### 4.2. Realtime PCR Based on OIE Protocol

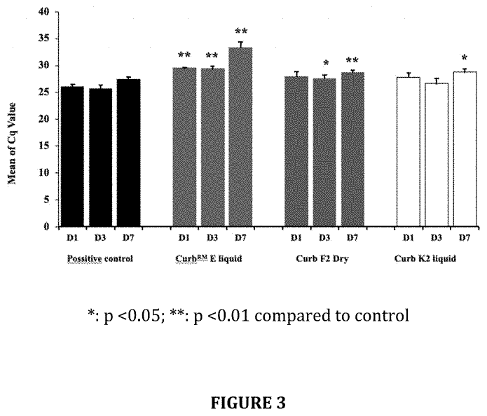

For detail, please see HAD and Cq values files (FIG. 3).

### 4.3. HAD50 Data

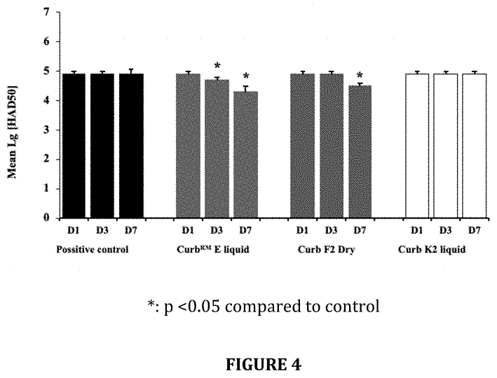

For detail, please see HAD and Cq values files (FIG. 4).

### V. Conclusion

Sal CURB RM E Liquid shown a strong inhibition of viricidal activity of ASFV isolated in Vietnam (realtime PCR from DPC1 to 7, HAD from DPC 3 to 7)
Sal CURB F2 Dry shown a significant inhibition of viricidal activity of ASFV from DPC3 to 7 based on realtime PCR and only DPC7 by HAD
Sal CURB K2 liquid shown only significant effect on DPC7 by realtime PCR. No effect was observed by HAD
ASFV can survive in feed up to 7days based on conventional PCR and realtime PCR

### EXAMPLE 2

### Examination of Kemin Sal Curb RME Liquid Inhibiting Viricidal Activity in Water

I. Information of ASF Strain Used in this Study
ASFVs isolated from died pigs in Ha Nam and Nam Dinh provinces in 2019

### II. Experiment Design

Water model: Fresh water, negative for ASFV.
Treatment of fresh water with Sal CURB RME Liquid before contamination with ASFV
   a. Control Groups
Positive control: fresh water treated with ASFV isolated in Vietnam at different doses: 10\hat{ }1HAD50 and 10\hat{ }3HAD50
Negative control: Fresh water (ASFV negative by real-time PCR)
   b. Experiment Groups
Fresh water treated with Sal CURB RME Liquid different doses at 3 kg, 1 kg, 0.75 kg, 0.5 kg/1000 litter (3 g, 1 g, 0.75 g and 0.5 g/1000 ml). Shaking the mix for 10 mins
The Sal Curb RME Liquid-treated water were exposed to ASFV at different doses of 10\hat{ }1HAD50 and 10\hat{ }3HAD50, respectively

### III. Sample Collection

Samples were collected at 30 min, 1 h, 2 h, 3 h, 12 h and 24 h after ASFV exposure and DNA extraction were performed immediately. DNA were stored at −20 for evaluation by real-time PCR.

TABLE 2
Sampling schedule for treatment and collection (10\hat{ }1HAD50)

| | Time | | 3 kg/1000 | 1 kg/1000 | 0.75 kg/1000 | 0.5 kg/1000 | Positive | Negative |
|---|---|---|---|---|---|---|---|---|
| Order | points | | litter | litter | litter | litter | Control | Control |
| 1 | 30 | min | 3 samples | 3 samples | 3 samples | 3 samples | 3 samples | 3 samples |
| 2 | 1 | h | 3 samples | 3 samples | 3 samples | 3 samples | 3 samples | 3 samples |
| 3 | 2 | h | 3 samples | 3 samples | 3 samples | 3 samples | 3 samples | 3 samples |
| 4 | 3 | h | 3 samples | 3 samples | 3 samples | 3 samples | 3 samples | 3 samples |
| 5 | 12 | h | 3 samples | 3 samples | 3 samples | 3 samples | 3 samples | 3 samples |
| 6 | 24 | h | 3 samples | 3 samples | 3 samples | 3 samples | 3 samples | 3 samples |

TABLE 3
Sampling schedule for treatment and collection (10\hat{ }3HAD50)

| | Time | | 3 kg/1000 | 1 kg/1000 | 0.75 kg/1000 | 0.5 kg/1000 | Positive | Negative |
|---|---|---|---|---|---|---|---|---|
| Order | points | | litter | litter | litter | litter | Control | Control |
| 1 | 30 | min | 3 samples | 3 samples | 3 samples | 3 samples | 3 samples | 3 samples |
| 2 | 1 | h | 3 samples | 3 samples | 3 samples | 3 samples | 3 samples | 3 samples |
| 3 | 2 | h | 3 samples | 3 samples | 3 samples | 3 samples | 3 samples | 3 samples |
| 4 | 3 | h | 3 samples | 3 samples | 3 samples | 3 samples | 3 samples | 3 samples |
| 5 | 12 | h | 3 samples | 3 samples | 3 samples | 3 samples | 3 samples | 3 samples |
| 6 | 24 | h | 3 samples | 3 samples | 3 samples | 3 samples | 3 samples | 3 samples |

### IV. Methods

Real-time PCR using OIE protocol

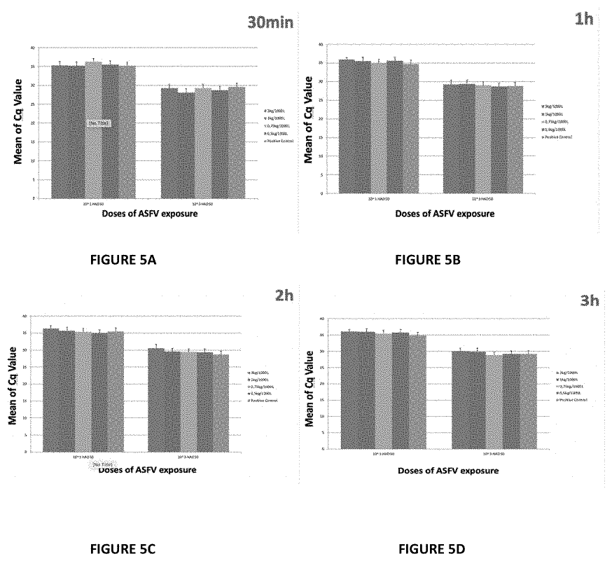

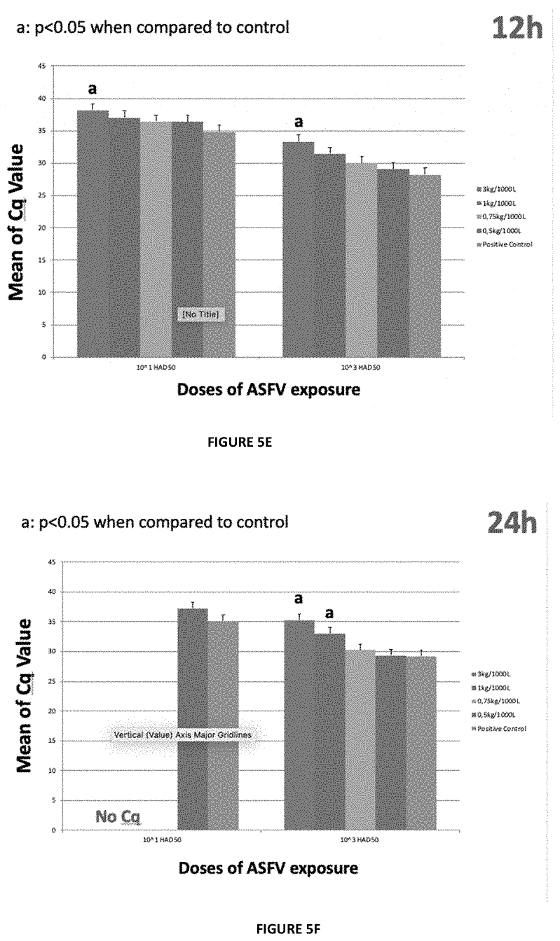

### V. Results (FIG. 5)

### VI. Conclusion

No effect of Sal CURB were observed at 30 min, 1 h, 2 h and 3 h after ASFV exposure when compared to control group
A significant effects was noted at 3 kg-treated group at 12 h after exposure to ASFV at both of doses of 10\hat{ }1HAD50 and 10\hat{ }3HAD50 when compared to control group (p<0.05); a modest enhance of Cq value was recognized in 1 kg-treated group at 12 h at both of 10\hat{ }1HAD50 and 10\hat{ }3HAD50
Sal CURB shown a strong antiviral effect after 24 h exposure in which at the dose of 3 kg, 1 kg and 0.75 kg/1000 litter, no Cq were noted in group spiked to 10\hat{ }1HAD50 of ASFV, a statistic effects were observed in the group exposed to 10\hat{ }3HAD50 at doses of 3 kg and 1 kg/1000 L of Sal CURB RME Liquid (p<0.05)

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplishes at least all of the intended objectives.

1. A composition for preventing and/or treating African Swine Fever (ASF) in swine comprising: at least one formaldehyde and; at least one organic acid.

2. The composition of claim 1, whereby the at least one organic acid is selected from the group consisting of acetic acid, propionic acid, butyric acid, citric acid, malic acid, tartaric acid, oxalic acid, lactic acid, formic acid, and benzoic acid.

3. The composition of claim 2 whereby the at least one organic acid is propionic acid and/or formic acid.

4. The composition of claim 1 further including an emulsifier.

5. The composition of claim 1, further including a pharmaceutically acceptable carrier.

6. The composition of claim 1, further including animal feed.

7. The composition of claim 6, whereby the composition is added to animal feed and includes at least 40 kg of the formaldehyde/metric ton of the animal feed, and at least 10 kg of the at least one organic acid/metric ton of the animal feed.

8. The composition of claim 7 whereby the composition includes at least 6 kg of an emulsifier/metric ton of the animal feed.

9. A method of preventing or treating African Swine Fever (ASF) in swine comprising: administering to swine a composition comprising a formaldehyde; and at least one organic acid.

10. The method of claim 9 whereby the composition whereby the at least one organic acid is selected from the group consisting of acetic acid, propionic acid, butyric acid, citric acid, malic acid, tartaric acid, oxalic acid, lactic acid, formic acid, and benzoic acid.

11. The method of claim 10 whereby the at least one organic acid is propionic acid and/or formic acid.

12. The method of claim 10, whereby the composition is administered to the swine by providing the composition in animal feed.

13. The method of claim 10, whereby the swine is a pig.

14. The method of claim 10, whereby the composition includes at least 40 kg of the formaldehyde/metric ton of the animal feed, and at least 10 kg of the at least one organic acid/metric ton of the animal feed.

15. The method of claim 14, whereby the composition further includes between about 6 kg of an emulsifier/metric ton of the animal feed.

16. The method of claim 10 whereby the composition is administered to the swine with a pharmaceutically acceptable carrier.

17. The method of claim 10 whereby the composition is administered to the swine through the swine's water source.

18. A method of manufacturing a composition for preventing and/or treating African Swine Fever (ASF) in swine comprising: combining a formaldehyde; and at least one organic acid to form a composition.
